(19) United States
(12) Patent Application Publication
(10) Pub. No.: US 2026/0147004 A1
(43) Pub. Date: May 28, 2026

(54) DEVICE, METHOD, AND APPARATUS FOR BIOLOGICAL TESTING WITH A MOBILE DEVICE

(71) Applicant: Emmanuel Mpock, Salida, CA (US)

(72) Inventor: Emmanuel Mpock, Salida, CA (US)

(21) Appl. No.: 19/178,066

(22) Filed: Apr. 14, 2025

Related U.S. Application Data
(60) Provisional application No. 63/633,208, filed on Apr. 12, 2024.

(51) Int. Cl. G01N 33/72; G01N 21/64; G01N 21/76; G01N 33/543

(52) U.S. Cl. CPC G01N 33/723; G01N 21/6428; G01N 21/6456; G01N 21/76; G01N 33/54333; G01N 33/726; G01N 2021/6439; G01N 2201/0221

(57) ABSTRACT

A method and system for performing assays for hemoglobin HbA1c, employing a smart phone that interacts with a disposable test strip coated with dry chemistry in physically and chemically segregated reactive zones. In the given regions of the strip, the chemistry interacts with the sample resulting in the conversion of the analyte, such as glycated hemoglobin, into a complexed substrate which can be quantified optically. The phone takes measurements of various parameters during the process of the test reactions, displays relevant instructions and the assay results.

### RELATIONSHIP TO OTHER APPLICATIONS

The present application claims the benefit of 63/633,208 filed 12 Apr. 2024, which is fully incorporated by reference herewith.

### Government support

None

### FIELD OF INVENTION

The field of the present invention relates to assay systems for HbA1c in a blood sample using a test strip (cassette)using a smart phone to analyze and quantify the amount of analyte in the blood sample.

### BACKGROUND

Glycated hemoglobin refers to a series of minor hemoglobin components that are formed through the attachment of glucose to the hemoglobin molecule. The human red blood cell is freely permeable to glucose. Within each red blood cell, glycated hemoglobin is formed at a rate that is directly proportional to the ambient glucose concentration. Approximately 97% of the total hemoglobin in circulating red blood cells is hemoglobin A. Hemoglobin A consists of four polypeptide chains, two a-chains and two b-chains. Glycation of the Hemoglobin A occurs through the covalent coupling of glucose with the N-terminal valine amino acid of each b-peptide chain. An unstable Schiff base (aldimine) is initially formed which then undergoes an irreversible Amadori rearrangement to form a stable ketoamine, Hemoglobin A1c (HbA1c).

The percentage of Hemoglobin A that is glycated to HbA1c is directly proportional to the time that red blood cells are exposed to glucose and to the average glucose concentration encountered. Measurement of the HbA1c fraction gives an integrated picture of the average blood glucose concentration during the half-life of the red cells, that is, over the last 60 days. The level of HbA1c is usually expressed as a percentage of total hemoglobin.

In normal subjects, HbA1c is typically in the range 3-6% of total hemoglobin. In patients with elevated glucose levels e.g. in the case of Type I and Type 2 diabetes, the level may rise to twice the upper limit of normal or more.

Long-term control of glucose levels in diabetics is very important. Too much glucose in the blood over many years can damage the eyes, kidneys and nerves. It also increases the risk for heart and blood vessel disease. The measurement of HbA1c as a percentage of total hemoglobin provides a valuable means of assessing the long-term control of glucose levels and also constitutes an important risk indicator for identifying Type 1 and Type 2 diabetics. This is supported and recommended by the United Kingdom Prospective Diabetes Study, the Diabetes Control and Complication Trial and the American Diabetes Association.

Quantitative and qualitative chemical assays are now accepted as very useful tools in the diagnostics industry for medical and food products. These tools in the hands of scientists have enabled diagnosis of various disease conditions, as well as in monitoring and management of patients undergoing various forms of therapy.

There is a need to provide a simple way to monitor the disease. Even more critically, there are hundreds of millions of individuals who are in a state known as prediabetes. In this stage, the disease can be reversed and the best global method for prediabetes screening is by HbA1c testing.

There is a need to develop an accurate, but inexpensive HbA1c test that can be easily and accurately performed anywhere in the world using a device which most of the people on earth have. This is a great opportunity to combat diabetes and slow its spread. Present methods utilize sophisticated and expensive equipment at considerable cost, in fixed locations. There is a need for small and portable handheld systems that can be used at the point of need and care, such as the doctor's office, clinic, patient's bedside or at home. The current invention aims to make testing at the point of care inexpensive, simple and accurate.

The present invention describes accurate and inexpensive, portable assays and test systems that provide flexibility, convenience and cost reduction. A key element for the success of these types of systems for optimal performance is ease of use, with few preparatory steps. As an example, a typical home self-test system of the invention used for blood analysis requires the patient to prick a finger with a sterilized lancet, apply a drop of blood sample and may be a buffer solution to the disposable strip, and then wait for the results. Assays that use other bodily fluids, such as urine essentially work in a similar manner. These devices are designed such that a typical lay person can perform the tests correctly with very little training.

### BRIEF DESCRIPTION OF THE INVENTION

The invention encompasses a method and system for performing assays for various target molecules, such as HbA1c in a sample, such as a blood sample. HbA1c is a glycated form of hemoglobin having glucose molecules non-enzymatically bound to it.

The method employs a smart phone with a camera, a screen and a CPU, all in functional communication with each other, mounted on a simple enclosed box (also called a “companion box”), where the camera is used to take a photograph of a reaction zone within a disposable test strip (strip) having microchannels (also called lumens) disposed therein, coated with dry chemistry in physically and chemically segregated reactive zones. This tests system provides the means of segregating analytes and then quantifying them independently as needed.

The test strip comprises chemistry that lyses the red blood cells, releasing all the forms of hemoglobin in the sample, including hemoglobin HbA1c.

In another embodiment, particularly for samples with extremely low analyte concentration, a reaction zone of the strip, is a reagent chemistry whereby certain reactants interact with the HbA1 analyte resulting in its conversion into a complexed substrate. This chemical specie produces light (chemiluminescence) that can be quantified optically when it interacts with another chemical specie in the sample path that is tailored for this specific chemical complexed substrate. This is a well-known art in the industry.

Reactions occur in the strip wherein boronic acid derivatives conjugated onto magnetic microparticles react with HbA1c to form chemical complexes which are permanently bound to the particles and are guided to defined locations by the prevailing magnetic field in functional communication with a chemiluminescent tag that can be quantified using optical measurement systems. The camera system is able to accurately and directly quantify the presence and absence of the HbA1c molecule.

The analyzer (in the smart phone) takes measurements at various desired optical wavelengths during the process of the test reactions, and displays relevant instructions, and then displays the assay results.

A general embodiment of the invention comprises the following process carried out in a test strip, where direct measurements of the various hemoglobin variants in a blood sample additively or by absence, and a smart phone, is used to detect and analyze said signal.
Providing a blood sample (“sample”);
Providing a test strip having a plurality of microchannels disposed therein, wherein said microchannels link a sample well with a reaction well;
Wherein the microchannels comprise at least one lysing agent (eg a non-ionic surfactant);
Wherein the test strip is adapted to comprise or interact with one or a plurality of magnets placed within or adjacent to the test strip, providing a magnetic field that extends functionally into one or more of the microchannels, sample well and/or reaction well;
Providing magnetic beads wherein boronic acid derivatives are attached to said magnetic beads;
placing the blood sample (which may be diluted or pre-diluted with a buffer) into the sample well of the test strip such that the sample is drawn into the microchannels by capillary action, wherein red blood cells in the sample are lysed by the lysing agent in the microchannels releasing free haemoglobin including HbA1c (glycated haemoglobin) which travels via the microchannels to the reaction well;
whereby, in use, the boronic acid derivatives attached to the magnetic beads react with HbA1c to form chemical complexes;
whereby when the Boronic acid derivatives react with HbA1c to form chemical complexes;
Wherein the light signal is detected and quantified by the analyzer (in the smart phone having a camera, a CPU and a screen, all in functional communication with each other) which displays the assay results on its screen.

### BRIEF DESCRIPTION OF THE DRAWINGS

The figures (Figs.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

FIG. 1 Schematic drawing of a microchannel showing sample port, treatment area and regions of interest (ROIs) in which a reaction is imaged by a camera.

FIG. 2 Schematic drawing of the test strip showing a sample port (oval) microchannels with areas of reagent placement (shown as a group of black dots) and regions of interest (ROIs) (rectangles bounded by dotted lines) in which a reaction is imaged by a camera. Multiple magnetic fields can be localized for isolated reactions. or the entire region of the strip can be subjected to a single field based on how many analytes are targeted.

FIG. 3 Schematic drawing showing structure of the test strip showing layered structure with a double sided adhesive layer into which capillary channels are cut, laminated between two clear films.

FIG. 4 Schematic drawing showing exemplary structure of the test strip with sample port shown as an oval.

FIG. 5 Schematic of the companion box with test strip and phone mounted onto it.

FIG. 6 Schematic drawing showing exemplary structure of the test strip with regions of interest shown as circles and sample port shown as an oval.

FIG. 7 Another view of the companion box with test strip and phone mounted onto it

FIG. 8 Graph of analyte concentration (X-axis) vs. optical response (Y-axis) showing linear relationship between analyte concentration (HbA1c) and optical signal.

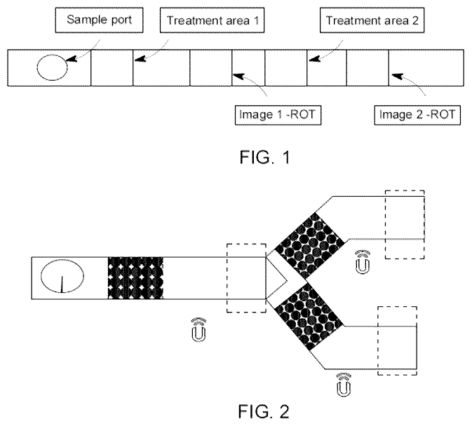

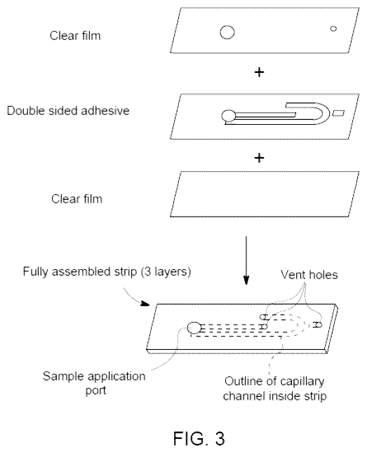

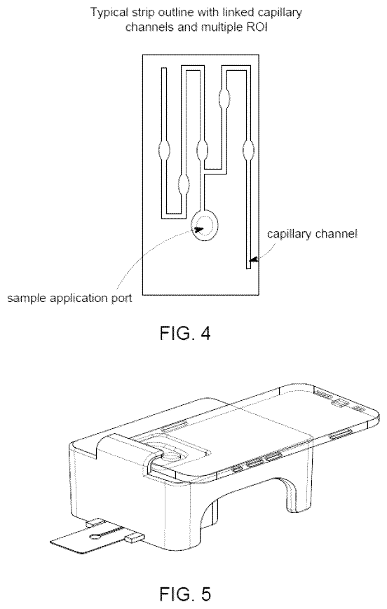

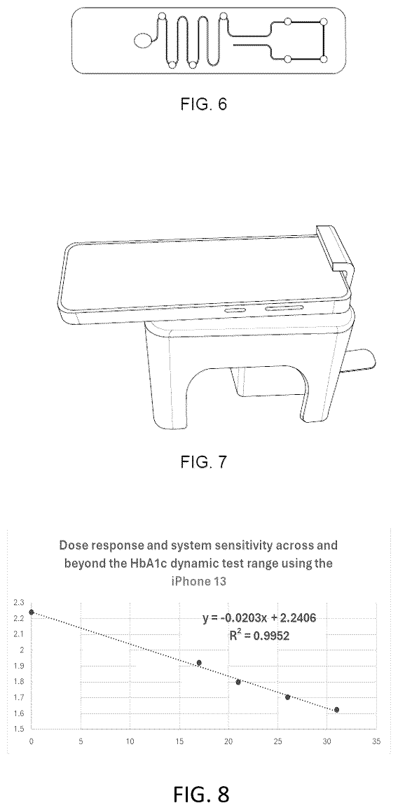

### DETAILED DESCRIPTION OF THE INVENTION

The invention encompasses systems, methods, and compositions for analyzing biological samples related to health conditions.

The method of the invention involves taking a blood sample, capturing an image with a mobile device, using analysis software on the device to analyze the image to identify and quantify its components within the sample.

The mobile device is generally a smart phone and may be referred to as the “electronic analyzer” as it is programmed with analytical software in the form of an application (app). Images are taken at pretreatment and during or after interaction with agents in the test strip, then analyzed via app image processing software on the mobile device.

The heart of the invention is a reaction performed on a strip having microchannels therein, wherein boronic acid compounds bound to magnetic micro-beads react with HbA1c in a blood sample to form chemical complexes which are bound thereto, that can then be localized using magnetic fields in the microchannels of the strip, and quantified using optical measurement systems with the aid of a companion box and analyzed and displayed using a smart phone.

The companion box is a simple partially enclosed box that is adapted to have a slot to receive the strip, and a location to hold and retain the phone so that the phone camera lens is suitably positioned to view a reaction chamber, and wherein the companion box, with the phone attached and the strip inserted, keeps out most or all ambient light, allowing optimum optical reading of the light signal at the reaction chamber.

In the reaction of the invention, boronic acid derivatives that are conjugated onto magnetic microparticles (beads) react with HbA1c from a blood sample to form molecular complexes. The HbA1c is captured through chemical bonding with the boronic acid derivatives.

Using positionable magnetic fields positioned at specific locations, the microparticles along with the captured molecular complexes with or without chemiluminescent tags can be directed to regions where they are optically quantified. Since a plurality of areas of interest (regions of interest) are monitored optically, the cameras are able to determine the optical colorimetric changes as the HbA1c is separated under the guidance of the magnetic field. The colorimetric changes are related to the HbA1cand hemoglobin concentrations, before capture, during capture, and after capture of the hemoglobin complexes. Using the cell phone app run by the CPU, these concentrations are calculated and reported as a percent of the total HbA1c and hemoglobin in the sample.

The microparticles can be other forms used in the industry like latex or any other micro beads, when the magnetic particles are encapsulated into the microparticles suitable for conjugation assays.

### Two Embodiments Emphasizing the Novelty of the Invention

The novelty and uniqueness of the invention is associated with its ability to cheaply, easily and efficiently detect the absence of the analyte of interest without an expensive, specialized machine, using existing commercially available smart phones. A sample is places in the test strip and lysed. An optical reading is taken of the “whole blood sample” at a first location. The sample is then reacted with molecules, bound to magnetic beads, which bind to the HbA1c molecules, thus removing the HbA1c molecules from the sample. The magnetic beads are retained using a magnet, and the remainder of the sample is moved to a second location and a second optical reading is taken of the sample. Thus the first reading and second reading are compared and analyzed to determine the total concentration of HbA1c in the sample.

Two main embodiments of the invention are as follows:
1. An assay system using a colorimetric signal
   (a) An unlysed whole blood sample is placed into a sample well of a test strip in which the sample well, or connecting microchannels are coated with lysing agent. Alternatively the sample may be pre-lysed before being deposited into the sample well of the test strip.
   (b) The blood travels down the microchannel by capillary action to Location 1.
   (c) A colorimetric reading (reading 1) is taken of “Total hemoglobin” (at Location 1).
   (d) The lysed blood travels down the microchannel and is contacted with a plurality of magnetic particles that are coated with a boronic acid derivative.
   (e) The boronic acid stable derivative interacts with the HbA1c (in a specific manner) to create a boronic acid-HbA1c complex (a “complex”), thereby immobilizing the HbA1c on the magnetic particles, which are localized within the test strip by magnets placed in or on or in proximity to the test strip at various set locations.
   (e) Optionally, a colorimetric reading (reading 2) may be taken at this stage of the immobilized complex comprising the bound HbA1c (at Location 2).
   (f) The lysed blood then travels further down the microchannel to a third reading zone where a colorimetric reading (reading 3) of the blood depleted of HbA1c is taken. We call this the “Depleted hemoglobin” reading (at Location 3).

The CPU of the smart phone runs a programme executing an algorithm that calculates the HbA1c concentration as a percentage of total hemoglobin.
2. An assay system using a Chemical signal (chemiluminescent or Fluorescent)
   (a) A sample (lysed or unlysed blood, serum, urine, saliva, biological, food or environmental extracts) is placed in sample well of a test strip and is drawn into the microchannels.
   (b) An analyte of interest reacts with a specific chemiluminescent or fluorescent tag (antibody, ligand or chemical) to form a complex to give a signal, and a reading (reading 1) is taken in Location 1.
   (c) The sample travels down the microchannel where the complex is specifically bound to magnetic particles conjugated with antibody or ligand to immobilize and concentrate the signal, and a reading (reading 2) is taken in Location 2.
   (d) The sample then travels down the microchannel to a third reading zone where a reading (reading 3) can be taken at Location 3.
   (e) The total signal (reading 1), the bound signal (reading 2) and the depleted signal (reading 3) are each measured directly.

The CPU of the smart phone runs a programme executing an algorithm that calculates the concentration of the analyte of interest.

A variation to this is linking the chemiluminescent or fluorescent tag to the magnetic particles to form the complex and the signal read in Location 2 where the analyte will be immobilized and concentrated. Readings can be taken in all 3 locations and algorithms will work out the concentration of the analyte.

### Other General Embodiments

In a typical embodiment a drop of blood is collected from a subject, (optionally mixed with a diluent buffer)and placed in a sample well in the strip. The blood sample is drawn into a flow path of microfluidic channel by capillary action. The microfluidic channels are coated in various locations with lysing reagents such as non-ionic surfactants, which lyse red blood cells (RBCs) and release the native non-glycated hemoglobin and HbA1c.

The HbA1c reacts with the boronic acid derivatives conjugated onto magnetic beads to produce a chemical complexes. A tag (chemiluminescent, fluorescent or colorimetric) is present either coupled to the magnetic beads or otherwise in functional communication with or interacting with the chemical complexes so produced. Upon formation of the chemical complex, a reaction occurs that provides a chemiluminescent, fluorescent or colorimetric signal.

The signal so produced will quantify the concentration of a species in the sample, where the intensity of light or color produced by the chemical reaction is proportional to the concentration of the species detected. The concentration of the substance detected is usually directly proportional to the intensity of light produced (in a chemiluminescent or fluorescent assay) or to the absorbance of light at a specific wavelength (in a colorimetric assay).

Images of specific areas within the flow path through the channels are obtained using a phone with a camera and a processor (CPU).

Images may be taken pre-treatment and during or after treatment and signals produced by interaction with agents in the test strip are then analyzed via app image processing software on the consumer device.

An exemplary embodiment is as follows:
(i) Providing a blood sample (“sample”);
(ii) providing a test strip having a plurality of microchannels disposed therein, wherein said microchannels link a sample well with a reaction well;
(iii) wherein the microchannels comprise at least one lysing agent (e.g. a non-ionic surfactant);
(iv) wherein the test strip is adapted to comprise or interact with one or a plurality of magnets placed within or adjacent to the test strip, providing a magnetic field that extends functionally into one or more of the microchannels, sample well and/or reaction well;
(v) providing magnetic beads wherein boronic acid derivatives are attached to said magnetic beads;
(vi) and optionally, wherein a chemiluminescent tag is attached to the magnetic beads or otherwise in functional communication with or interacting with the chemical complexes so produced;
(vii) placing said beads within the test strip in one or more of the microchannels, sample well and/or reaction well;
(viii) placing the blood sample (which may be diluted or pre-diluted with a buffer) into the sample well of the test strip such that the sample is drawn into the microchannels by capillary action, wherein red blood cells in the sample are lysed by the lysing agent in the microchannels releasing free haemoglobin including HbA1c (glycated haemoglobin) which travels via the microchannels to the reaction well;
(ix) whereby, in use, the boronic acid derivatives attached to the magnetic beads react with HbA1c to form chemical complexes;
(x) whereby when the Boronic acid derivatives react with HbA1c to form chemical complexes, the chemiluminescent tag (optional) or colorimetric tag produces a light signal;
(xi) wherein the light signal is detected and quantified by the analyzer in the smart phone having a camera, a CPU and a screen, all in functional communication with each other, which displays the assay results on its screen.

In another embodiment the invention comprises A method for performing assays, like the measurement of hemoglobin A1c in a whole blood sample in a disposable strip with a micro-capillary system coated with dry chemistry, whereby the sample is lysed in or outside the test strip, and the amount of total hemoglobin automatically measured optically in a disposable test strip, while the sample is in a dynamic state in motion. Reaction and filtration zones contain compounds that can specifically form chemical complexes with the HbA1c that can be easily quantified. The design allows isolation, chemical and physical segregation and separation of the target complexes whereby they can be optically measured and quantified; whereby the amount of individual complexes generated is directly correlated to the amount of A1c. The percent of A1c in the sample is the quantified amount as a percentage of quantified total hemoglobin in the sample. The method performs assays with microliter volumes, in a disposable cartridge coated with dry chemistry. When the test sample is applied into the cartridge, it is subjected to chemical interaction, after which the cartridge automatically executes chemical and physical segregation and separation of the target analytes, either individually or as complexes which can be optically measured and quantified. The invention includes systems whereby the analytes can be quantified using a smart phone and just the cartridge, where the volume of the sample is in micro liter volumes, and a method and system whereby the cartridge is able to control flow in multiple directions, without external pumps.

The method of the invention is comparable to ion exchange liquid chromatography in which the pretreated samples pass through chemically modified resin to trap a specific biological component in the sample, which is later released by changing the composition of the eluting solution.

In the present invention, optically clear channels on a plastic test strip consist of regions of varying chemical treatment. The invention enables whole complexes to be kept static by reducing the surface energy chemically of the capillary channels resulting in immobilization of target chemistries and reaction block, and only released during chemical interaction, which allow the biological sample to exist in pretreated state and after treatment state.

Specifically, the microchannels of the test strips are coated with modified resin magnetic particles (microspheres of different resins encapsulate the magnetic particles).

The method as described herein further comprises the steps of inserting the test strip into an adaptor linked to the consumer device, wherein the consumer device comprises a digital camera, and optionally provides a GPS location, an image storage memory.

Further the method also comprises steps of providing test results and electronic media to health providers, along with service recommendations to the consumer device.

The capillary channels are the same as the flow channels cut from double sided adhesive and laminated on both sides with plastic film of a desired resin type. The chemistry is dried down in these channels.

The device of the invention comprises a test strip and a camera holder (also called housing, box, or companion box) adapted to hold a smart phone in such a position that the lens of the smart phone can focus on the target area of the disposable cartridge to take a reading.

The test strip is a single-use strip that can include agents that interact with a biological sample, such as blood, serum, plasma, or sputum. It may also test non-biological samples like water or chemical species. The test strip can have one or more plastic films made of materials like nylon or polypropylene. Additionally, it might feature an optical barcode or RFID tag. The background may include a random colored pattern for security, calibration, and validation, interpreted by an algorithm processing signals from a digital camera.

A single-use test strip is designed with at least one sample port region to facilitate the application of biological sample. The sample port may be coated with specific reagents such as preservatives and stabilizers, for biological sample. Sample treatment methods may employ combinations of buffers, and reagents for lysing cellular membranes.

Treatment regions may contain combinations of buffers, reagents for lysing cellular membranes, and magnetic particles conjugated with immunoglobin, enzymes or non-protein agents like clot preventing agents commonly used in the industry.

The region of interest is void of any chemistry associated with the sample port or treatment areas.

The Companion box (also called “holder” or “camera housing” or ‘housing”) is designed to securely hold a test strip and a mobile phone. When secured to the phone, the combined device acts as a portable tabletop device. The box The Companion box may have any number of geometries for easy placement of to a wide variety of make and models of mobile phones.

The Companion boxes able to securely hold the mobile phone in place and substantially blocks out ambient light. The mobile phone is placed on and fixed to the companion box to provide a uniform optical path between the mobile phone's illumination sources and the mobile phone camera. This increases measurement repeatability and voids reading errors due to illumination and field-of-view variations or tilts, all of which are substantially eliminated with the designs disclosed herein. The Companion box includes rail guides specifically designed for securely receiving the test strip (strip, test strip), which can be adjusted or configured to accommodate various types of test strips. The rail guide is precisely dimensioned to secure the test strip in a fixed position, ensuring that the relevant sections of the test strip are accurately aligned for imaging by the mobile phone's camera. Additionally, the rail guide features windows at predetermined locations to allow the camera to capture images of the pertinent areas of the test strip.

A plurality of magnetic fields will be present or can be induced at specific locations in the channels of the test strip. These magnetic fields are used to control the mobility and other related movement of the microparticles.

A magnetic field generator (and electromagnet) is provided within close proximity of the strip, and can be moved to one or more specific areas of interest in the strip. This magnetic field can be generated using electromagnets or permanent magnets made from materials with inherent magnetic properties, such as iron, cobalt, nickel, and certain alloys like Alnico and NdFeB.

In another embodiment the invention provides a tool for performing for safety tests for food products and other purposes where identification and quantification of a chemical species is required.

A preferred embodiment is a method and system for performing assays using a disposable cartridge (also called a strip or sample strip) that fits into a companion box that allows proper positioning between the disposable cartridge and the optical lens of the smart phone (electronic analyzer). The smart phone contains and uses optical electronics systems for analyte quantification which is run on software using a smart phone.

The smart phone is placed on a bespoke companion box. The companion box is designed such that the disposable cartridge fits into a slot in the box, and is securely held in such as position that the lens of the smart phone can focus on the target area of the disposable cartridge to take a reading.

The disposable cartridge has a sample port, a serpentine microfluidic channel and at least one reaction chamber. The microfluidic channel and/or reaction chamber is coated with dry chemistry components in physically and/or chemically segregated reactive zones whose interactions with the sample results in optically quantifiable reaction. The dry chemistry components interact with the sample resulting in the conversion of an analyte, such as glycated hemoglobin, into a complexed substrate which can be quantified optically.

The system takes measurements of various parameters during the process of the test reactions, like color intensities of the strip, reacting samples at determined wavelengths, and displays relevant instructions and the final results using a smart phone.

The present invention provides a cartridge that fits into a companion box. The smart phone also fits onto the companion box and is held stably in place such as position that the lens of the smart phone can focus on the target areas of the disposable cartridge.

The smart phone comprises an electronic analyzer system (app) for performing assays and for determining the quantity of target analytes in the given test sample.

Typically, the sample is deposited directly into the sample well (also referred to as sample receptacle, or sample port) of the cartridge, or alternatively is mixed with a diluent buffer and then the deposited into the sample well.

Microchannels are fabricated using techniques like photolithography, soft lithography, micro-milling, laser ablation, and 3D printing.

Between the vent holes and sample port are a series of microchannels into which reagents are deposited. These are termed ‘reaction zones’. Vent holes allow for microfluidic flow between any two linked points in the capillary channel (standard capillary flow requirement).

In selected regions (specific areas where lysing is required-not all areas may necessarily need lysing)of the channels, agents that lyse the cells can be placed, lysing red blood cells and releasing hemoglobin. The lysing agents may be provided along a part of the channels or along the entire length of the channels, and are, for example, nonionic surfactants, such as for example TRITON™ X-100. These are responsible for lysing the red blood cells to release the different variants of Hemoglobin into the capillary channel. The system of the invention separates and quantifies the HbA1c hemoglobin from the other types of hemoglobin found in blood and measures its concentration.

With the flow of the sample into the different reaction zones, the different phases (non lysed blood, lysed blood, blood with and without HbA1c) of the reactions are monitored optically segregated by the designed chemical filtering process This is just a fancy term to describe the removal of captured of chemically captured or bound hemoglobin from one area to another and measurements are made whereby a determination of the total Hemoglobin is made. The results can then be expressed as the percent of HbA1c present in the sample which is then displayed on the display window of the smart phone. At the end of the test, as determined by the imaging system of the smart phone, it displays a message for the completion of the test.

The strip can be made from a suitable material that is selected for its properties, such as chemical inertness to blood and all associated chemical reactions, optical clarity and high surface energy to facilitate blood flow along the capillaries.

For purposes of illustration, this embodiment of the invention is described by reference to a disposable strip (or ‘strip’) formed by joining two or more solid supports such as plastic film or glass. Moreover, the solid support is such that a contained free flowing stream of liquid flows into its segmented lumen (the capillary channels), whereby the segregation is within the module is the entire independent strip piece capable of quantifying clusters (Clusters are regions that may be monitored simultaneously for optical changes resulting from the underlying chemical reactions) of molecular complexes in the reaction zones of the strip. The lumen can be any geometric shape, and are preferably circular or rectangular, with dimensions that are predetermined to provide cavity volumes sufficient to hold the samples and to allow for the reactions to occur. Thus, the lumen can have a height spanning between about 0.01 mm to 1.0 mm and depending on the length and width of the support material, can have a length of about 150 mm or shorter based on sample volume desired. The width of the channels can vary from 0.5 mm to 2 mm. Both dimensions need to stay within the industry known dimensions that allow for capillary forces to exist and can be easily determined by anyone skilled in the art.

The disposable strip can be placed in the slot in the companion box on which the smart phone is securely placed such that the camera lens faces the reaction well in the strip. An app in the smart phone will guide the user through the test. The detection mechanism is not limited to just a smart phone since any optical detection methods could also be used.

The heart of the invention is a reaction wherein compounds react with HbA1c to form chemical complexes that can be quantified using optical measurement systems.

An example system for automated detection for use with the present disposable strip and associated methods comprises an excitation source, a monochromator (or any device capable of spectrally resolving light components, or a set of narrow band filters) and a detector array. The excitation source may comprise infrared, blue or UV wavelengths and the excitation wavelength can be shorter than the emission wavelength(s) to be detected.

The applied sample is accurately distributed into the various reaction zones via the capillary channels. The positioning of the reaction zones can be such that independent reactions can occur in the various reaction zones even though they share a common sample from the same pool.

In addition, antibodies and related antigens can be employed to perform similarly. The antibody can be a monoclonal or polyclonal antibody (Ab), or Ab fragment containing the antigen binding site, or complementarity determining region (CDR), such as an F(ab′)₂ or Fab fragment.

The detectable moiety or label may be a radioactive, fluorescent or chemiluminescent moiety, or an enzyme. Alternatively, a labeled-second Ab which recognizes the species specific Fc fragment of the first Ab may also be used. Further, the antibody may be labeled with a detectable label such as in an ELISA.

When using a fluorescent marker in a biological sample, the fluorescent marker will only emit light at a different wavelength when it absorbs light of a specific wavelength called the excitation wavelength. Thus the invention may comprise a light emitting source that emits light at a specific excitation wavelength or may comprise excitation filters used to isolate the specific excitation wavelength from a broad-spectrum light source (like a white light lamp). Different fluorophores have different excitation and emission wavelengths, so the light source and filters must be matched to the fluorophore being used. For example green fluorescent protein (GFP) is typically excited by blue light and emits green light, while other fluorophores like DAPI require UV excitation.

A detailed embodiment of the invention is as follows. A method for measuring the concentration of HbA1c in a blood sample, comprising the steps of:
(i) providing a blood sample (“sample”);
(ii) providing a test strip having a plurality of microchannels disposed therein, wherein said microchannels link a sample well with a reaction well (a) wherein the microchannels comprise at least one lysing agent (e.g. a non-ionic surfactant); and (b) wherein the test strip is adapted to comprise or interact with one or a plurality of magnets placed within or adjacent to the test strip, providing a magnetic field that extends functionally into one or more of the microchannels, sample well and/or reaction well;
(iii) providing magnetic beads wherein boronic acid derivatives are attached to said magnetic beads; wherein a chemiluminescent tag is attached to the magnetic beads or otherwise in functional communication with or interacting with the chemical complexes so produced; wherein said beads are located within the test strip in one or more of the microchannels, sample well and/or reaction well;
(iv) placing the blood sample (which may be diluted or pre-lysed and diluted with a buffer) into the sample well of the test strip such that the sample is drawn into the microchannels by capillary action; wherein red blood cells in the sample are lysed by the lysing agent in the microchannels releasing free haemoglobin including HbA1c (glycated haemoglobin) which travels via the microchannels to the reaction well; whereby, in use, the boronic acid derivatives attached to the magnetic beads react with HbA1c to form chemical complexes; and whereby, when the Boronic acid derivatives react with HbA1c to form chemical complexes, and a chemiluminescent tag produces a light signal; wherein the light signal is detected and quantified by the analyzer (in the smart phone having a camera, a CPU and a screen, all in functional communication with each other, which displays the assay results on its screen.

The method of further comprising the steps of inserting the test strip into a housing, the housing adapted to receive the test strip and a mobile phone comprising a camera so that the test strip and mobile phone are stably held in relation to each other, such that the housing blocks out the majority of any ambient light, and such that the camera is positioned to view and take an image of the reaction well of the test strip.

The method wherein the mobile phone comprises a CPU, a memory means, a digital camera and a screen all in functional contact with each other, and a GPS location system.

The method wherein the mobile phone stores and transmits test data to an individual or a health care provider.

The method wherein the method measured total hemoglobin, and the measurement of the total hemoglobin and HbA1c are direct, wherein the system is able to eliminate the HbA1c from the sample and again measure the residual hemoglobin content of the sample.

The method wherein red blood cells in the blood sample are lysed in the strip while in transit through the microchannels and wherein the sample is moved through the microchannels by capillary forces and fractionated into two factions, wherein one faction is without the Hba1c hemoglobin variant and quantified directly.

Depending on the analyte to be detected and quantified, the design, length, width of the microchannels can be varied.

Another embodiment is a method further comprising providing a companion box wherein said companion box is a partially enclosed box having a slot adapted to receive a test strip, and a window adapted to attach and hold a smart phone with a camera lens, so that the camera lens is positioned to view a reaction chamber on the test strip, and wherein the companion box, with the phone attached and the strip inserted, excludes most or all ambient light, allowing optimum optical reading of the light signal at the reaction chamber; and attaching said smart phone to the window of the companion box; and inserting said test strip into said slot of said companion box, and taking one or more optical readings selected from reading 1, reading 2 and reading 3.

In a related embodiment the distance of the camera lens to the reaction chamber may be greater than 4 inches. The window to which the smart phone may be attached is between 1 inch and 2.5 inches wide and about 1 inch and 3 inches long. The smart phone may be placed at a horizontal or an angled position on the companion box. The companion box may be at least partially textured in the area where the smart phone is attached to the window to increase stability of the attachment. The inside of the companion box may be black or of a dark color. The companion box is made from recyclable materials. The companion box may be made of a material selected from the group consisting of paper, injection molded plastic, and wood.

While the invention has been particularly described with reference to a preferred embodiment and various alternate embodiments, it will be understood by persons skilled in the relevant art that various changes in form and details can be made therein without departing from the spirit and scope of the invention.

### GENERAL DISCLOSURES

The specification incorporates by reference all documents referred to herein and all documents filed concurrently with this specification or filed previously in connection with this application, including but not limited to such documents which are open to public inspection with this specification. All numerical quantities mentioned herein include quantities that may be plus or minus 20% of the stated amount in every case, including where percentages are mentioned. As used in this specification, the singular forms “a, an”, and “the” include plural reference unless the context clearly dictates otherwise. Thus, for example, a reference to “a part” includes a plurality of such parts, and so forth. The term “comprises” and grammatical equivalents thereof are used in this specification to mean that, in addition to the features specifically identified, other features are optionally present. For example, a composition “comprising” (or “which comprises”) ingredients A, B and C can contain only ingredients A, B and C, or can contain not only ingredients A, B and C but also one or more other ingredients. The term “consisting essentially of” and grammatical equivalents thereof is used herein to mean that, in addition to the features specifically identified, other features may be present which do not materially alter the claimed invention. The term “at least” followed by a number is used herein to denote the start of a range beginning with that number (which may be a range having an upper limit or no upper limit, depending on the variable being defined). For example, “at least 1” means 1 or more than 1, and “at least 80%” means 80% or more than 80%. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Where reference is made in this specification to a method comprising two or more defined steps, the defined steps can be carried out in any order or simultaneously (except where the context excludes that possibility), and the method can optionally include one or more other steps which are carried out before any of the defined steps, between two of the defined steps, or after all the defined steps (except where the context excludes that possibility). When, in this specification, a range is given as “(a first number) to (a second number)” or “(a first number)-(a second number)”, this means a range whose lower limit is the first number and whose upper limit is the second number. For example, “from 40 to 70 microns” or “40-70 microns” means a range whose lower limit is 40 microns, and whose upper limit is 70 microns.

### Definitions

As used herein, the term “subject” encompasses mammals and non-mammals. Examples of mammals include, but are not limited to, any member of the Mammalian class. Examples of non-mammals include, but are not limited to, birds, fish and the like.

“Label”. As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, ligands (e.g., biotin, avidin, streptavidin or haptens) and the like.

“Solid Support”. As used herein, a “solid support” refers to a solid surface such as a plastic plate, magnetic bead, latex bead, microtiter plate well, glass plate, nylon, agarose, acrylamide, nitrocellulose and the like.

“Specific” in reference to the binding of two molecules or a molecule and a complex of molecules refers to the specific recognition of one for the other and the formation of a stable complex as compared to substantially less recognition of other molecules and the lack of formation of stable complexes with such other molecules. Exemplary of specific binding are antibody-antigen interactions, enzyme-substrate interactions, polynucleotide hybridizations and/or formation of duplexes, cellular receptor-ligand interactions, and so forth.

“Companion box” (also called “frame” or “closed frame” or “camera housing” or ‘housing”) refers to a box sealed at least partially against light, having a window into which a strip may be inserted and having a placement area for a mobile phone. The Companion box includes rail guides specifically designed for securely receiving the test strip (strip, test strip), which can be adjusted or configured to accommodate various types of test strips. The rail guide is precisely dimensioned to secure the test strip in a fixed position, ensuring that the relevant sections of the test strip are accurately aligned for imaging by the mobile phone's camera. Additionally, the rail guide features windows at predetermined locations to allow the camera to capture images of the pertinent areas of the test strip.

“Strip” (aka reaction strip, test strip) refers to the disposable rectangular device adapted to accept a sample and to perform various chemistry reactions within the strip.

“Boronic acid derivatives” refer to various molecular structures derived from the boronic acid molecule.

“Flow Path” Refers to the Path Along Which a Liquid Flows Within the Microchannels of a test strip.

“Microfluidic channel” or “microchannel” or “lumen” refers to any channel with dimensions of less than 1.0 mm in diameter. A microchannel will allow the flow of liquids in by capillary action, not affected by gravity flow.

A “Treatment area” refers to Areas specifically treated to allow for certain behavior of the reagents and samples like flow or immobilization

A “Region of interest (ROI)” refers to Region where measurements are made

‘Coated’ means a property of a surface where a substance is physically attached to a surface.

A “chemiluminescent tag” is a molecule or substance that emits light as a result of a chemical reaction, often used in immunoassays and Western blotting to detect and visualize specific proteins or other molecules.

A “chemical complex” is a Molecule or moiety formed from a combination of 2 or more molecules

A “sample port” refers to an area or a volume into which a sample may be deposited.

A “treatment area” refers to an area in the strip where a sample can be subjected to a target treatment like lysing of the cells in the sample prior to moving to another region where a lysed sample is needed

A “reaction well” refers to areas targeted for certain specific chemical reactions

A “vent hole” refers to a hole located somewhere along the strip that links the capillary channel and the external atmosphere

A “modified resin magnetic particle” refers to a non-magnetic resin microparticle encapsulating a magnetic particle that can be influenced by a magnetic field

A “solid support” refers to any stationary surface to which a molecule may be conjugated to

A “phone” or “smart phone” or “mobile phone” or “cell phone” refers to a device that comprises a CPU, a memory means, a digital camera with an optical flash and a screen all in functional contact with each other, and optionally a GPS location system. Where a phone etc. is mentioned, any other suitable device can equally be used and no telephonic or communication function is required or implied. The “phone” is used only to take a photograph and process data using a CPU and display said data on a screen.

“Magnet: includes electromagnets, and when it is stated that such magnets may be placed on, within or adjacent to the test strip, providing a magnetic field that extends functionally into one or more of the microchannels, this essentially means that the magnets may be placed at any location suitable to magnetically hold the magnetic beads of the invention.

1. A method for measuring the concentration of HbA1c in a blood sample, comprising the steps of:
(i) providing a blood sample;
(ii) providing a test strip having a plurality of microchannels disposed therein, wherein said microchannels link at least a sample well with a one or more reaction wells, wherein (a) the reaction well or the microchannels comprise at least one lysing agent; and wherein (b) the test strip is adapted to comprise or interact with one or a plurality of magnets placed on, within or adjacent to the test strip, providing a magnetic field that extends functionally into one or more of the microchannels, sample well and/or reaction well;
(iii) providing magnetic beads wherein boronic acid derivatives are attached to said magnetic beads; wherein said beads are located within the test strip in one or more of the microchannels, sample well and/or reaction well, and localized to a desired location by the use of the magnetic fields;
(iv) placing a whole or pre-lysed blood sample into the sample well of the test strip such that the sample is drawn into the microchannels by capillary action; wherein unlysed red blood cells in the sample are lysed by the lysing agent, releasing haemoglobin including HbA1c at a first location (Location 1);
(v) taking an optical reading (reading 1) at Location 1;
(vi) causing the sample to travel through the microchannel towards a second location (Location 2) wherein said magnetic beads with boronic acid derivatives attached thereto contact the sample and whereby the boronic acid derivatives interact with HbA1c in said sample to form a stable boronic acid-HbA1c complex, thereby immobilizing the HbA1c on the magnetic beads;
(vii) optionally, taking an optical reading (reading 2) at Location 2;
(viii) while maintaining the magnetic beads at Location 2 by use of the magnets, causing the remainder of the sample to travel through the microchannel towards a third location and taking an optical reading (reading 3) at this location (Location 3);
(iv) comparing at least reading 1 with reading 3 to provide direct test data;
(v) analyzing said test data using the CPU of a smart phone executing a programme that calculates the HbA1c concentration as a percentage of total hemoglobin, and
(vi) displaying the resultant data on a screen of the smart phone.

2. The method of claim 1 further comprising the steps of inserting the test strip into a companion box, the companion box adapted to receive the test strip and a smart phone comprising a camera so that the test strip and mobile phone are stably held in relation to each other, such that the companion box blocks out the majority of any ambient light, and such that the camera is positioned to view and take an image of various areas of the test strip.

3. The method of claim 2 wherein the smart phone comprises a CPU, a memory means, a digital camera and a screen all in functional contact with each other, and a GPS location system.

4. The method of claim 2 wherein the smart phone stores and transmits test data to an individual or a health care provider.

5. The method of claim 1 wherein the method measures total hemoglobin, and the measurement of the total hemoglobin and HbA1c are direct colorimetric, chemiluminescent or fluorescent measurements, wherein the system is able to separate the HbA1c-containing fraction from the sample by complexing the HbA1c with boronic acid derivatives attached to magnetic beads, and again measure the residual hemoglobin content of the sample.

6. The method of claim 1 wherein red blood cells in the blood sample are lysed in the strip while in transit through the microchannels and wherein the sample is moved through the microchannels by capillary forces and fractionated into two factions, wherein one faction is enriched in HbA1c and one fraction has a reduced concentration of HbA1c or no HbA1c, and the HbA1c content is quantified directly.

7. A method for quantifying a analyte of interest contained in a sample comprising: placing a sample of lysed or unlysed blood, serum, urine, saliva, biological material, food or an environmental extract into a sample well in a test strip whence the sample is drawn into a microchannel by capillary action to a first location (Location 1) where the analyte of interest reacts with a chemiluminescent or fluorescent tag to form a complex, thereby emitting a signal for which a reading is taken (reading 1 at Location 1); causing the sample to travel through the microchannel wherein the analyte of interest can be bound to a second species to form a complex that is bound to magnetic particles conjugated with antibody or ligand to immobilize and concentrate the signal (reading 2 at Location 2); causing the sample, now depleted of the analyte of interest, to further travel through the microchannels to a third read zone where a reading can be taken (reading 3 at Location 3); wherein the total signal, the bound signal and the depleted signal can be measured directly.

8. The method of claim 7 using a chemiluminescent or fluorescent tag is linked to the magnetic particles to form the complex and a signal is read in Location 2 where the analyte of interest is immobilized and concentrated.

9. The method of claim 1 further comprising providing a companion box wherein said companion box is a partially enclosed box having a slot adapted to receive a test strip, and a window adapted to attach and hold a smart phone with a camera lens, so that the camera lens is positioned to view a reaction chamber on the test strip, and wherein the companion box, with the phone attached and the strip inserted, excludes most or all ambient light, allowing optimum optical reading of the light signal at the reaction chamber; and attaching said smart phone to the window of the companion box; and inserting said test strip into said slot of said companion box, and taking one or more optical readings selected from reading 1, reading 2 and reading 3.

10. The method of claim 9 wherein, in use, the distance of the camera lens to the reaction chamber is greater than 4 inches.

11. The method of claim 10 wherein, in use, the window to which the smart phone is attached is between 1 inch and 2.5 inches wide and about 1 inch and 3 inches long.

12. The method of claim 10 wherein, the smart phone is placed at a horizontal or an angled position on the companion box.

13. The method of claim 10 wherein the companion box is at least partially textured in the area where the smart phone is attached to the window to increase stability of the attachment.

14. The method of claim 10 wherein the inside of the companion box is black or a dark, light-absorbing color.

15. The method of claim 10 wherein the companion box is made from recyclable materials.

16. The method of claim 15 wherein the companion box is made of a material selected from the group consisting of paper, injection molded plastic, and wood.

17. The method of claim 9 further comprising providing a light emitting source that emits light at a specific excitation wavelength, or a broad-spectrum light source and one or more excitation filters.
